(19) United States
(12) Patent Application Publication
(10) Pub. No.: US 2025/0002969 A1
(43) Pub. Date: Jan. 2, 2025

(54) DISCRIMINATION OF ANTIBIOTIC RESISTANT AND SENSITIVE SAMPLES

(71) Applicants: University of Pittsburgh - Of the Commonwealth System of Higher Education, Pittsburgh, PA (US); Duquesne University of the Holy Spirit, Pittsburgh, PA (US)

(72) Inventors: John Alston Kellum, JR., Pittsburgh, PA (US); Robert Hugh Edgar, Pittsburgh, PA (US); Cierra Lynn White, Jones Mills, PA (US); John Andrew Viator, Pittsburgh, PA (US); John D. Hempel, North Braddock, PA (US)

(21) Appl. No.: 18/684,769

(22) PCT Filed: Aug. 20, 2022
(86) PCT No.: PCT/US2022/040978

§ 371 Date: Feb. 19, 2024

Related U.S. Application Data
(60) Provisional application No. 63/235,406, filed on Aug. 20, 2021.

(51) Int. Cl. C12Q 1/18; C12N 7/00; G01N 15/01; G01N 15/10; G01N 15/1434; G01N 29/24

(52) U.S. Cl. CPC C12Q 1/18; C12N 7/00; G01N 15/01; G01N 15/1434; G01N 29/2418; G01N 2015/1006; G01N 2291/02466

(57) ABSTRACT

A method of determining antibiotic resistance of bacteria includes dividing a sample including the bacteria into a plurality of sub-samples, mixing labeling: agents with each of the plurality of sub-samples. Each of the labeling agents is detectible via a detection system including a sensor to detect photoacoustic waves. The labeling: agents are further active to selectively bind with the bacteria. The method further includes applying an antibiotic to one or more of the plurality of sub-samples and not to one or more others of the plurality of sub-samples, characterizing at least one of the sub-samples to which the antibiotic has been applied and at least one of the sub-samples to which no antibiotic has been applied after a determined period of time via the detection system, and determining antibiotic resistance of the bacteria on the basis of the characterization via the detection system.

### CROSS-REFERENCE TO RELATED APPLICATIONS

This application claims benefit of U.S. Provisional Patent Application Ser. No. 63/235,406, filed Aug. 20, 2021, the disclosure of which is incorporated herein by reference.

### GOVERNMENTAL INTEREST

This invention was made with government support under grant no. CA182840 awarded by the National Institutes of Health. The government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Antimicrobial resistance has become one of the most pressing concerns for global health and therefore has become an ever increasing focus of research and product development. Antibiotic resistance arises most often when bacteria gain and express gene cassettes that confer the ability to outmaneuver the action of an antibiotic. Bacteria employ two main strategies to gain resistance to antibiotics; pumps and enzymes. Pumps work to evacuate the antibiotic from cell cytoplasm before they can reach a critical level. Enzymes work to degrade antibiotic molecules before they can have an effect. Non-genetic antibiotic resistance also occurs, though in a minority of cases, and is often mediated by small molecule communication between cell populations or general impermeability of the cell wall. Resistance to antibiotics is an inevitable outcome of their use. The first cases of penicillin resistance were reported only two years after its widespread use. There are multiple pathways of acquiring antibiotic resistance in each type of bacteria. Horizontal gene transfer is the dominant method of acquiring antibiotic resistance.

The most rapid test for antibiotic resistance in Staph. aureus is a polymerase-chain-reaction-based or PCR-based test for the mecA gene. PCR is much faster than the standard antibiotic disc method, but is used in only a minority of cases. The antibiotic disc method requires an antibiotic disc placed on a lawn of bacteria and measuring the zone of inhibition caused by the antibiotic. This method requires an additional 24 hours after initial bacterial identification. PCR methods can be completed in 3-4 hours but can suffer from failure arising from unknown sample conditions and concentrations or amplification of sub-populations that mask clinically relevant genetic traits or markers. Most problematic for clinicians are the false negatives when a resistant strain is misidentified as a susceptible strain. The resultant delay in appropriate antimicrobial treatment can be detrimental to patients. Occasionally, these tests give intermediary or conflicting results regarding a strain's resistance.

Isolates that are heterogeneous in their expression of a resistance gene can lead to serious problems for patient treatment. A low expressing strain will appear to be susceptible when using the antibiotic disc method and when treated with a first-line antibiotic such as oxacillin. In some cases, the majority of bacteria are killed, and the small remaining percentage are dealt with by the immune system. In other cases, the infection appears to be cleared only to reemerge a few days later. Although the infection remains a result of the original bacterial strain with which the patient first presented, the infection will be treated by the clinician as an unresolved infection or a new infection, depending on the time taken for the infection to reemerge. The reemergence of infection will likely be classified as a hospital-acquired infection. Hospital-acquired infections are no longer reimbursed by the Centers for Medicare and Medicaid Services. Additionally, increased hospital-acquired infections negatively impact each institution's Hospital-Acquired Condition score resulting in overall reduction of funding and reimbursement rates from Medicare and Medicaid Services. For healthcare institutions the misclassification is a costly mistake since they can no longer charge for the initial, community-acquired infection.

Flow cytometry has been effectively used to analyze large heterogeneous cell populations since Wolfgang Gohde first developed it in 1968. Fluorescent flow cytometry relies on the absorption of laser light by an object and the detection of the fluorescence from that object at an alternate wavelength. Fluorescence flow cytometry has a number of disadvantages that can limit its usefulness for bacterial identification. Light, though very powerful, is easily quenched or blocked in a turbid environment such as blood. Additionally, small amounts of light from single cells can be hard to detect in dilute samples. Fluorescent flow cytometry works best with large numbers of cells and clear non turbid environments. Photoacoustic flow cytometry (PAFC), however, relies on the absorption of laser light and the detection of ultrasound waves created by the photoacoustic effect.

The photoacoustic effect has been used in a number of manners. For example, depth profiling in human tissues for the treatment of port wine stains has been done as well as photoacoustic imaging of blood vessels. PAFC has successfully been used to enumerate circulating tumor cells and has been shown to be a robust predictor of metastasis in melanoma. Additionally, PAFC has been used successfully for the isolation of circulating tumor cells by several groups. PAFC was developed to find rare, individual particles in complex environments. The ultrasonic waves created by the photoacoustic effect are robust and not quenched in turbid media, such as cell suspensions or blood samples. Recently, PAFC has been used on blood samples, in vitro, to detect and identify bacteria using modified bacteriophage as optical tags. Edgar, R. H. et al . . . “Bacteriophage-mediated identification of bacteria using photoacoustic flow cytometry,” Journal of Biomedical Optics 24(11), p. 115003 (2019) and U.S. Pat. Nos. 10,544,443 and 10,961,557, the disclosures of which are incorporated herein by reference.

There is a need for tests that can differentiate between antibiotic susceptible and antibiotic resistant bacteria in clinical samples as well as differentiating between heterogeneous antibiotic resistant bacteria.

### SUMMARY

In one aspect, a method of determining antibiotic resistance of bacteria includes dividing a sample including the bacteria into a plurality of sub-samples, mixing labeling agents with each of the plurality of sub-samples. Each of the labeling agents is detectible via a detection system including a sensor to detect photoacoustic waves. The labeling agents are further active to selectively bind with the bacteria. The method further includes applying an antibiotic to one or more of the plurality of sub-samples and not to one or more others of the plurality of sub-samples, characterizing at least one of the sub-samples to which the antibiotic has been applied and at least one of the sub-samples to which no antibiotic has been applied after a determined period of time via the detection system, and determining antibiotic resistance of the bacteria on the basis of the characterization via the detection system. Characterization via the detection system may, for example, include quantifying the bacteria in the at least one of the sub-samples to which the antibiotic has been applied and the at least one of sub-samples to which no antibiotic has been applied after the determined period of time. In a number of embodiments, determining antibiotic resistance of the bacteria includes characterizing a difference between the quantification of the bacteria in the at least one of the sub-samples to which the antibiotic has been applied and the quantification of the bacteria in the at least one of sub-samples to which no antibiotic has been applied after the determined period of time.

In a number of embodiments, determining antibiotic resistance of the bacteria includes making a determination if the bacteria is resistant to the antibiotic or homogeneous susceptible to the antibiotic. In a number of embodiments, determining antibiotic resistance of the bacteria includes making a determination if the bacteria is homogeneous resistant, heterogeneous resistant or homogeneous susceptible to the antibiotic.

In a number of embodiments, the labeling agent includes an entity which is active to selectively bind to the bacteria. The labeling agent may, for example, include a protein (for example, an antibody or a host-recognition protein), a peptide, a bacteriophage, or a portion of a bacteriophage active to selectively bind to a bacteria (for example, a portion or section of a bacteriophage including one or more host-recognition proteins.) The bacteriophage may, for example, be modified to delay or eliminate lysing of a bacterial cell, while retaining activity active to selectively bind to a species of bacteria. Portions of bacteriophage suitable for use herein include, for example, a bacteriophage ghost, a section (for example, tail) of a bacteriophage from which at least a portion of the capsid had been removed, a portion of a bacteriophage (for example, a section of a tail, filament, or other portion of a bacteriophage including host-recognition/attachment functionality), or a host-recognition protein. In a number of embodiments, a portion of a bacteriophage or bacteriophage portion is that part of the bacteriophage remaining after modification to remove of part of or all of the nucleic acid therefrom such that lysing is delayed or eliminated as compared to the complete bacteriophage as described further below.

In a number of embodiments, the protein is an antibody or a host-recognition protein. In a number of embodiments, the portion of a bacteriophage includes or is a bacteriophage ghost, a bacteriophage from which at least a portion of a capsid thereof has been removed, a section of a bacteriophage including one or more host-recognition proteins, or a bacteriophage host-recognition protein. The portion of a bacteriophage may, for example, be a bacteriophage from which at least a portion of the capsid thereof has been removed (for example, a bacteriophage tail) or a section of a bacteriophage including one or more host-recognition proteins (for example, a section of a tail or a filament).

In a number of embodiments, the labeling agents include one or more labels that is/are detectible via the detection system attached to the entity active to selectively bind to the bacteria (for example, a protein, a peptide, a bacteriophage, or a portion of a bacteriophage active to selectively bind with the bacteria. The one or more labels may, for example, include a compound that is detectible via the detection system attached to the entity active to selectively bind to the bacteria (for example, attached to a protein, a peptide, a bacteriophage, or a portion of a bacteriophage). The one or more labels may, for example, include a solid particle that is detectible via a detection system attached to the entity, wherein the solid particle is between 1 nm and 500 μm in size, 10 nm and 500 μm in size, or 10 nm and 100 μm in size. In a number of embodiments, the labeling agent comprises a solid particle attached to a plurality of entities active to selectively bind to the bacteria. In a number of embodiments, a plurality of the entities are attached to the solid particle. The plurality of entities may, for example, include a plurality of portions of a bacteriophage.

A sufficient number of labeling agents may, for example, be mixed with the bacteria so that at least one labeling agent is bound to each cell of the bacteria. In a number of embodiments, a sufficient number of the labeling agents is mixed with the bacteria so that a plurality of labeling agents is bound to each cell of the bacteria.

The antibiotic may, for example, include an aminoglycoside, a carbapenems, acephalosporins, a quinolone, a fluoroquinolone, a glycopeptide, a lipoglycopeptide, a macrolide, an oxazolidinones, a penicillin, a polypeptide, a rifamycin, a sulfonamide, a streptogramins, a tetracyclines, chloramphenicol, clindamycin, daptomycin, fosfomycin, lefamulin, metronidazole, mupirocin, nitrofurantoin, or tigecycline.

In another aspect, a labeling agent includes one or more labels that are detectible via the detection system including a sensor to detect photoacoustic waves attached to at least a portion of a bacteriophage which has been modified to delay or eliminate lysing of a bacterial cell and is active to selectively bind to a species of bacteria. In a number of embodiments, the one or more labels are attached to a portion of a bacteriophage, at least a portion or part of bacteriophage nucleic acid is absent from the portion of a bacteriophage. The portion of a bacteriophage may, for example, include or be a bacteriophage ghost, a bacteriophage from which at least a portion of a capsid thereof has been removed, a section of a bacteriophage including one or more host-recognition proteins, or a host-recognition protein. The portion of a bacteriophage may, for example, be a bacteriophage from which at least a portion of the capsid thereof has been removed (for example, a bacteriophage tail) or a section of a bacteriophage including one or more host-recognition proteins (for example, a section of a tail or a filament). As described above, the protein may, for example, be an antibody or a host-recognition protein.

The one or more labels may, for example, include a compound that is detectible via the detection system attached to the entity active to selectively bind to the bacteria (for example, attached to a protein, a peptide, a bacteriophage, or a portion of a bacteriophage). The one or more labels may, for example, include a solid particle that is detectible via a detection system attached to the entity, wherein the solid particle is between 1 nm and 500 μm in size, 10 nm and 500 μm in size, or 10 nm and 100 μm in size. In a number of embodiments, the labeling agent comprises a solid particle attached to a plurality of entities active to selectively bind to the bacteria. In a number of embodiments, a plurality of the entities are attached to the solid particle. The plurality of entities may, for example, include a plurality of portions of a bacteriophage. The solid particle may be inherently detectible via the detection system or may include one or more entities (for example, groups or compounds) that are detectible via a detection system.

In another aspect, a method of labeling bacteria for detection via a photoacoustic detection system including a sensor to detect photoacoustic waves includes attaching to the bacteria a labeling agent comprising one or more labels that are detectible via the detection system. The one or more labels are attached to at least a portion of a bacteriophage which has been modified to delay or eliminate lysing of a bacterial cell and is active to selectively bind to a species of bacteria or to a portion of a bacteriophage which is active to selectively bind to a species of bacteria.

In a number of embodiments, the one or more labels are attached to a portion of a bacteriophage, wherein at least a part of or a portion of bacteriophage nucleic acid is absent from the portion of a bacteriophage. The portion of a bacteriophage may, for example, include or be a bacteriophage ghost, a bacteriophage from which at least a portion of a capsid thereof has been removed, a section a bacteriophage including one or more host-recognition proteins, or a host-recognition protein. The portion of a bacteriophage may, for example, be a bacteriophage from which at least a portion of the capsid thereof has been removed (for example, a bacteriophage tail) or a section of a bacteriophage including one or more host-recognition proteins (for example, a section of a tail or a filament).

As described above, the one or more labels may, for example, include a compound that is detectible via the detection system. The one or more labels may, for example, include a solid particle that is detectible via a detection system, wherein the solid particle is between 1 nm and 500 am in size, 10 nm and 500 μm in size, or 10 nm and 100 μm in size. In a number of embodiments, a plurality of the bacteriophage or a plurality of the portions of the bacteriophage are attached to the solid particle. In a number of embodiments, a plurality of the portions of a bacteriophage are attached to the solid particle. The solid particle may be inherently detectible via the detection system or may include one or more entities (for example, groups or compounds) that are detectible via a detection system.

In another aspect, a method of determining antibiotic resistance of bacteria includes determining that a species, strain, or type of bacteria is present in a sample, after determining that the species, strain, or type of bacteria is present in the sample, mixing a labeling agent including a label that is detectible via a detection system including a sensor to detect photoacoustic waves with the sample, wherein the labeling agent is active to selectively bind with bacteria of the species, strain or type of bacteria that are resistant to an antibiotic, and determining antibiotic resistance of the species, strain, or type of bacteria on the basis of characterization via the detection system. Characterization via the detection system may include quantifying the bacteria in the sample to which the labeling agent is bound.

In a number of embodiments, determining that the species, strain, or type of bacteria is present in the sample includes mixing a speciating labeling agent including a label that is detectible via the detection system. The speciating labeling agent is active to selectively bind with the species, strain or type of bacteria. The method further includes using the detection system to determine the presence of the speciating labeling agent bound to the species, strain, or type of bacteria.

In a further aspect, a composition includes an entity which selectively binds to a first bacteria and a solid particle attached to the entity in a manner so that the entity retains the ability to selectively bind to the first bacteria. The solid particle is detectible via a detection system including a sensor to detect photoacoustic waves, and wherein the solid particle is between 1 nm and 500 μm in size, 10 nm and 500 μm in size, or 10 nm and 100 μm in size. The solid particle may be inherently detectible via the detection system or may include one or more entities that are detectible via the detection system.

The entity which selectively binds to the first bacteria may, for example, include a protein, a peptide, a bacteriophage, a bacteriophage which has been modified to delay or eliminate lysing of a bacterial cell and which is active to selectively bind to a species of bacteria, or a portion of a bacteriophage which is active to selectively bind to a species of bacteria. In a number of embodiments, the entity which selectively binds to the first bacteria includes a bacteriophage, a bacteriophage which has been modified to delay or eliminate lysing of a bacterial cell and which is active to selectively bind to a species of bacteria, or a portion of a bacteriophage which is active to selectively bind to a species of bacteria. The entity which selectively binds to the first bacteria may, for example, be or include a portion of a bacteriophage which is selected from the group of a bacteriophage ghost, a bacteriophage from which at least a portion of the capsid thereof had been removed, a section a bacteriophage including a host-recognition protein, and a host-recognition protein of a bacteriophage.

In a number of embodiments, a plurality of the entities are attached to the solid particle. The plurality of entities may, for example, include a bacteriophage, a bacteriophage which has been modified to delay or eliminate lysing of a bacterial cell and which is active to selectively bind to a species of bacteria, or a portion of a bacteriophage which is active to selectively bind to a species of bacteria as described above. In a number of embodiments, each of the plurality of entities includes a portion of a bacteriophage selected from the group consisting of a bacteriophage ghost, a bacteriophage from which at least a portion of the capsid thereof had been removed, a section a bacteriophage including a host-recognition protein, and a host-recognition protein of a bacteriophage. The portion of a bacteriophage may, for example, be selected from the group consisting of a bacteriophage from which at least a portion of the capsid thereof had been removed, a section a bacteriophage including a host-recognition protein, and a host-recognition protein of a bacteriophage.

In a number of embodiments, the composition further includes at least one other entity, different from the first entity, which selectively binds to a second bacteria, different from the first bacteria, attached to the particle. The composition may, for example, further include a plurality of the at least one other entity which selectively binds to a second bacteria, different from the first bacteria, attached to the microparticle.

In a number of embodiments, the other entity comprises a protein, a peptide, a bacteriophage, a bacteriophage which has been modified to delay or eliminate lysing of a bacterial cell and which is active to selectively bind to a species of bacteria, or a portion of a bacteriophage which is active to selectively bind to a species of bacteria. In a number of embodiments, the other entity includes a bacteriophage, a bacteriophage which has been modified to delay or eliminate lysing of a bacterial cell and which is active to selectively bind to a species of bacteria, or a portion of a bacteriophage which is active to selectively bind to a species of bacteria.

The present devices, systems, methods, and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a transmission electron microscope or TEM micrograph of bacteriophage Det7 virion particles with tail sheath, capsid, and tail fibers labeled, wherein the TEM micrograph was taken at a magnification of 52,000× using a Leica TEM with uranyl acetate stain.

FIG. 2 illustrates a photomicrograph of multiple T4 bacteriophage particles attached to a single E. coli cell imaged using Helium Ion microscopy taken from Leppänen et al., “Imaging bacterial colonies and phage-bacterium interaction at sub-nanometer resolution using helium-ion microscopy,” Advanced Biosystems 1(8), p. 1700070 (2017) and schematic illustrations of bacteriophage particles attached to a bacteria cell, a labeled bacteriophage, a labeled bacteriophage ghost, and a labeled tail portion of a bacteriophage with the capsid completely removed, wherein photoacoustic labels internal to the capsid in the bacteriophage ghost are illustrated in grayscale.

FIG. 3 illustrates schematically an embodiment of a photoacoustic flow chamber with ports labeled for identification in a system hereof.

FIG. 4A illustrates a study of untreated cultures and treated cultures of a clinical isolate exhibiting susceptibility to daptomycin, wherein the upper, left-hand panel (a) shows optical density (OD) for 2 hours growth differentiation, the upper right-hand panel (b) shows optical density for the full 16 hours of growth, and the center panel (c) shows cell count via photoacoustic detection of the sample tested at a two-hour timepoint, wherein complete inhibition of growth and zero detection in treated samples is observed.

FIG. 4B illustrates a study of untreated cultures and treated cultures of a clinical isolate exhibiting resistance to daptomycin, wherein the upper, left hand panel (a) shows optical density for 2 hours growth differentiation, the upper right-hand panel (b) shows optical density for the full 16 hours of growth, and the center panel (c) shows cell count via photoacoustic detection of the sample tested at a two-hour timepoint, wherein no delay or inhibition of growth is observed and both treated and untreated samples reach exponential growth simultaneously.

FIG. 4C illustrates a study of untreated cultures and treated cultures of a clinical isolate (heterogeneous strain A) exhibiting heterogeneous resistance to daptomycin, wherein the upper, left hand panel (a) shows optical density for 2 hours growth differentiation, the upper right-hand panel (b) shows optical density for the full 16 hours of growth, and the center panel (c) shows cell count via photoacoustic detection of the sample tested at a two-hour timepoint, wherein a delay of exponential growth is observed.

FIG. 4D illustrates a study of untreated cultures and treated cultures of another clinical isolate (heterogeneous strain B) exhibiting heterogeneous resistance to daptomycin, wherein the upper, left hand panel (a) shows optical density for 2 hours growth differentiation, the upper right-hand panel (b) shows optical density for the full 16 hours of growth, and the center panel (c) shows cell count via photoacoustic detection of the sample tested at a two-hour timepoint, wherein no delay in initial growth is observed but severe retardation of growth starting at 1.5 hours and continuing until the end of the test are observed.

FIG. 5 illustrates tubes containing ghost particles with Direct Red (or DRED) and propidium iodide (ProID) (G3), ghost particles with DRED and ethidium bromide (EthBr) (G2), and ghost particles with DRED (G1).

FIG. 6A illustrates a single functionalized microsphere with multiple bound phage tails in random orientations.

FIG. 6B illustrates multiple functionalized microspheres with attached phage tails.

FIG. 7 illustrates absorbance spectrum of microspheres functionalize with red and blue dye.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a label” includes a plurality of such labels and equivalents thereof known to those skilled in the art, and so forth, and reference to “the label” is a reference to one or more such labels and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”. “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

10048| Bacterial resistance continues to be a growing problem worldwide. The 2019 antibiotic resistance threat report from the CDC estimates over 2.3 million antibiotic resistant infections occur in the United States each year. Bacteremia is a serious and potentially lethal condition. Staph. aureus is a leading cause of bacteremia, and methicillin resistant Staph. aureus (MRSA) accounts for more than a third of the cases. Compared to methicillin sensitive Staph. aureus, MRSA is more than twice as likely to be fatal. Furthermore, sub-populations of seemingly isogenic bacteria may exhibit a range of antibiotic susceptibilities, often called heterogenous resistance. These heterogeneous antibiotic resistant infections are often misdiagnosed as hospital acquired secondary infections because there are no clinically used tests that can differentiate between homogeneous and heterogeneous antibiotic resistance. Homogeneous susceptibility, homogeneous resistance, and heterogeneous resistance or heteroresistance are, for example, discussed in Dewachter, L. et al., “Bacterial Heterogeneity and Antibiotic Survival: Understanding and Combatting Persistence and Heteroresistance,” Molecular Cell 76, 255-267 (2019), the disclosure of which is incorporated herein by reference. In a number of embodiments of devices, systems, and methods hereof, rapid bacterial identification is achieved using photoacoustic flow cytometry and labeled bacteriophages with the characterization and differentiation of antibiotic resistant and susceptible bacterial infections.

In photoacoustic flow cytometry, pulsed laser light is delivered to a sample flowing past a focused transducer and particles that absorb laser light and create an acoustic response thereto. Optically labeled bacteriophage may, for example, be added to a bacterial mixture that flows through the photoacoustic chamber. The presence of target bacteria is determined by bound, labeled phage which are detected photoacoustically.

In a number of representative studies hereof, incubation of bacterial samples in the presence and absence of the antibiotic daptomycin created a difference in bacterial cell quantification (that is, cell count, cell mass, optical density, etc.), which is, for example, quantified using photoacoustic flow cytometry. The bacterial cells are combined with a labeling agent which binds with the bacterial that includes a photoacoustically responsive tag, label, or dye. As described above, the photoacoustic tags, labels or dyes of the labeling agent create or emit detectible acoustic/sound or pressure waves upon the absorption of light. Such acoustic/sound or pressure waves are detectible using, for example, a detection system including a photoacoustic sensor.

As further described above, to develop a representative system of determining antibiotic resistance, the representative antibiotic daptomycin was used in conjunction with photoacoustic flow cytometry and bacteriophage as molecular labeling or tagging agents in a number of studies hereof. Daptomycin has shown a lack of cross-resistance with other antibiotic classes as well as being broadly active against MRSA isolates.

Bacteriophage are viruses that infect bacteria in a specific manner and may be used for classifying bacterial strains. A bacteriophage's ability to discriminate and selectively bind tightly to their host bacteria is vital to their fitness and evolutionary survival. Even in complex environments, bacteriophage are able to identify and bind target bacteria within seconds. Bacteriophage-host attachment is achieved via protein-protein interactions with the long tail fibers or tail spike proteins.

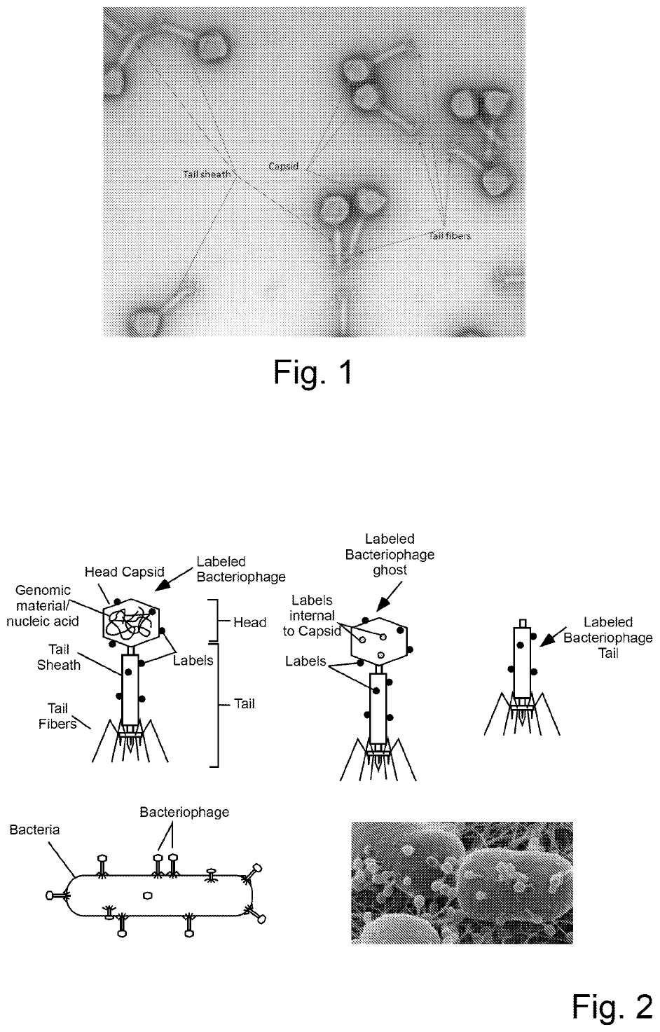

Those proteins have developed to be among the most stable protein complexes found in nature. Although a number of entities, including proteins such as antibodies, can be used to attach to bacterial cells as bacterial probes in the methods hereof, host-recognition proteins of bacteriophage (found, for example, on tails or filaments of bacteriophage, depending upon the type of bacteriophage) have many advantages over, for example, antibodies. Bacteriophage including host-recognition proteins in, for example, tail fibers or filaments, are produced as part of the bacteriophage self-replication within a bacterial host, making them less expensive to produce than antibodies. An electron micrograph of bacteriophage including tail fibers is shown in FIG. 1. Additionally, bacteriophage or portions/sections of bacteriophage including host-recognition proteins are more stable than antibodies. In general, a portion of a bacteriophage or bacteriophage portion is that portion of the bacteriophage remaining after removal of a portion of all of the nucleic acid therefrom such that lysing is delayed or eliminated as compared to the complete bacteriophage as described further below. Host-recognition proteins of a bacteriophage bind to a specific receptor. Bacteriophage tail fibers and filaments have greater specificity than antibodies and have evolved to bind to essential surface antigens of the bacteria and are therefore hard for the bacteria to change. Bacteriophage have evolved alongside bacteria as they have differentiated into new subspecies of bacteria, even those that have acquired antibiotic resistance. Bacteriophage attaching to bacterial surface antigens are displayed photographically and schematically in FIG. 2.

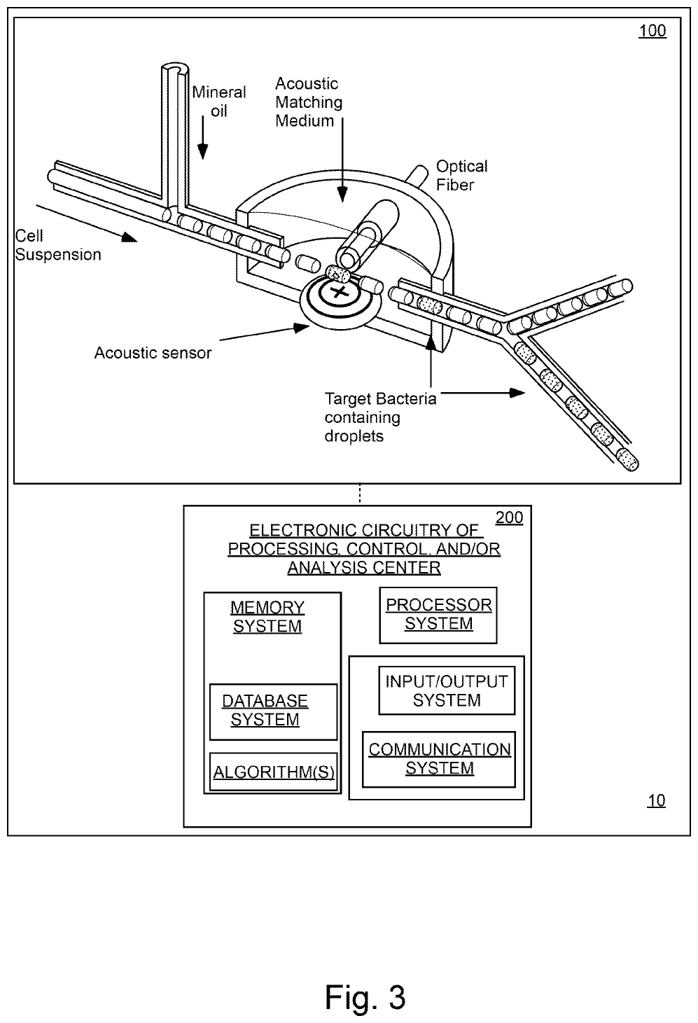

An additional advantage when used in connection with a PAFC system 100 (see FIG. 3) provided by bacteriophage is that size plays an important role. In that regard, single free-floating phage particles are typically or selectively below the detection threshold of a photoacoustic system. Bacteriophage attach to outer surface antigens on the surface of bacterial cells allowing for multiple bacteriophage to bind to a single cell. Regarding the number of bacteriophage attaching to an individual cell, Max Delbrück demonstrated in the 1930's that bacteriophage binding to surface receptors followed a pattern where the vast majority of bacterial cells will have a full complement of bacteriophage bound, and very few will have more or less bound. When labeled bacteriophage (or labeled, selectively-binding portions thereof) aggregate on the surface of bacterial cells, they form a sufficiently large optical absorber to create a detectable acoustic response. As a result, acoustic signals may be detected only from target bacteria of the bacteriophage of interest and not from unbound bacteriophage, depending upon the sensitivity of the photoacoustic detection system. In a number of representative studies, for example, a threshold was set for the acoustic signal amplitude at 2.5 times the noise floor, meaning that even bacterial cells with far fewer bacteriophage attached create a detectable and quantifiable signal.

Additionally, the bacterial cell suspension may be diluted so that the expected value of cells in the detection volume is one, following a Poisson distribution. Assuming a uniform distribution of cells from a well-mixed sample, the vast majority of detections will have a single bacterial cell, though a few might have two. A negligible number will have three or more. With a simple amplitude threshold detection, the method hereof is not dependent on relative numbers of attached bacteriophage, bacteriophage portions or other selective binding entity, as long as there are enough to reach threshold. An automated classifiers for the photoacoustic signals may be generated via characterization of a sufficiently large data pool.

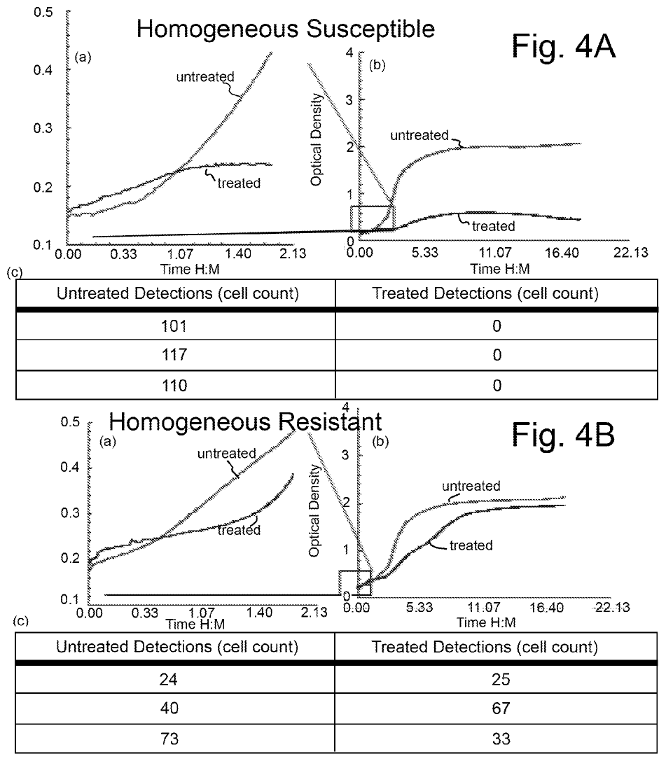

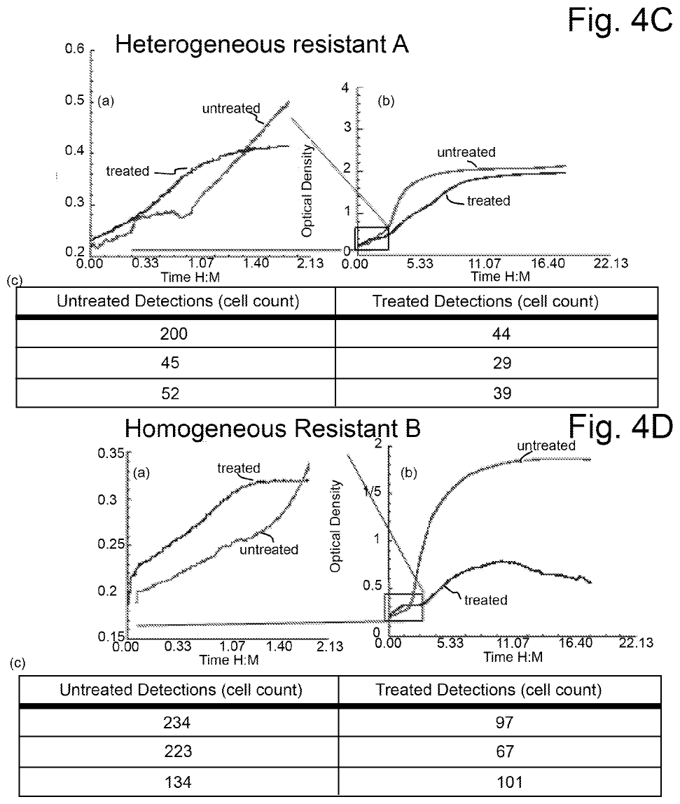

Growth curves of multiple clinical isolates of the representative bacteria S. aureus as illustrated in FIGS. 4A through 4D were determined using a BioTek Synergy HI plate reader (available from BioTek Instruments (of Agilent Technologies), Winooski, Vermont). S. aureus isolates growth rates were tested in the presence and absence of the representative antibiotic daptomycin. Differential growth rates were identifiable after two hours of the sixteen-hour growth curve. Two-hour differentiation was confirmed by Newton-Raphson root finding method.

Samples treated with daptomycin fell into three categories: resistant, susceptible, and heterogeneous resistant. Resistant strains are those wherein no inhibition of growth was observed in the treated sample versus the untreated control. In resistant strains, the rate of exponential growth was approximately identical between treated and untreated samples as well as nearly identical carrying capacity. Susceptible strains are those which show nearly complete inhibition of growth in treated sample versus untreated control.

Heterogeneous resistant strains are those wherein clonal isolates (genetically identical) growth curves where intermediary to susceptible and resistant growth curves. Heterogeneous samples displayed, for example, either a delay in reaching exponential growth phase or a complete retardation in achieving exponential growth. All samples were tested using the PAFC system in parallel to measuring growth rates. Growth curves for each of the bacterial strains were matched with their reported genotype. Strains that showed susceptibility at 0.25 μg/ml of daptomycin matched genotypically with MRSA strains where the mecA gene was not present in the genome.

In a number of studies, SP1 bacteriophage were grown using Staph. aureus strain SAI 13 (available from ATCC, Old Town Manassas, Virginia) and concentrated using methods described in R. H. Edgar, et al., “Bacteriophage-mediated identification of bacteria using photoacoustic flow cytometry,” Journal of Biomedical Optics 24(11), p. 115003, 2019 and in U.S. Pat. Nos. 10,544,443 and 10,961,557. Purified phage of 1×10¹² plaque forming units per milliliter (PFU/ml) were added to a saturated solution of Direct Red 81 dye (available from Sigma Aldrich, Saint Louis, Missouri). Virion particles were then pelleted and resuspended in buffer (10 mM Tris, pH 7.5, 10 mM MgCl₂, 68 mM NaCl). This process was repeated to ensure the removal of unbound dye. The absorbance spectrum of dyed phage was determined using the BioTek HI and compared to that of undyed phage particles. Dyed phage were titered to ensure no detrimental effects were observed from the dying process. Dyed phage were retested for their ability to infect after 150 days and no difference in titer was observed.

All strains were tested in the presence and absence of daptomycin. In a number of studies hereof, cultures were diluted into fresh media and regrown for 2 hours in the presence and absence of 25 μg/ml of daptomycin. Each sample was then incubated at room temperature for 10 minutes with multiple dyed phage per bacterial cell. Incubated samples were then processed through PAFC system 100 and number of detected cells recorded. Isolates were tested in triplicated for both the plate reader and PAFC system. Number of cells (panels (c)) detected are displayed with corresponding growth curves (panels (a) and (b)) in FIGS. 4A through 4D.

As set forth above, in the studies of FIGS. 4A through 4D, four clinical isolates were tested in the presence and absence of daptomycin. Photoacoustic events for each isolate were recorded and compared to growth curves. Once again, samples treated with the representative antibiotic daptomycin fell into three categories: susceptible (FIG. 4A), resistant (FIG. 4B), and heterogeneous resistant (FIGS. 4C and 4D). In FIGS. 4A through 4D, untreated culture and treated culture growth curves are labeled. In each figure, the left-hand panel (a) shows 2 hours growth differentiation, panel (b) shows a full 16 hours of growth, and panel (c) shows cell counts determined via photoacoustic detections of each sample tested at a two-hour time point.

Comparison of growth curves for each bacterial strain over 16 hours using a BioTek HI plate reader in connection with photoacoustic characterization provided confirmation of reliable differentiation between strains growth at 2 hours. Daptomycin has been found to be more broadly active against MRSA isolates than the standard oxacillin. As described above, daptomycin has shown a lack of cross-resistance with other antibiotic classes. Sensitivity to daptomycin is dosage dependent for both MRSA (MIC 0.25-1 μg/ml) and VRSA (MIC >4 μg/ml). Daptomycin was used at a concentration of 0.25 μg/ml as has been used in several studies and has been widely effective against antibiotic resistant Staph. aureus strains tested throughout Europe.

Growth curves for each of the bacterial strains were matched with their reported genotype. Strains that showed susceptibility at 0.25 μg/ml of daptomycin matched genotypically with MRSA strains where the mecA gene was not present in the genome. In the presence of daptomycin, little or no growth was observed in homogeneous susceptible strains at either the 2 hour or 16-hour time point as can be seen in FIG. 4A. To correlate the optical density or OD growth curves with photoacoustic response, multiple strains were tested in the PAFC system in the presence and absence of daptomycin. Dyed bacteriophage SP1 was added to resuspended cultures to add optical absorption to the bacterial cells for photoacoustic detection. For homogeneous susceptible strains, no bacteria were detected in the treated bacterial cultures demonstrating a complete inhibition of growth as seen in FIG. 4A. As described above, the PAFC system results were corroborated by the 16-hour growth curves demonstrating the ability to identify antibiotic sensitivity in under 4 hours.

Once again, resistant strains fell into two distinct categories. What is defined herein as homogeneous resistant strains were strains in which the growth in the presence and absence of daptomycin was indistinguishable. Examples of homogeneous resistant strains are shown in FIG. 4B. Homogeneous resistance could also be described as having complete penetrance of mecA gene expression. When tested with PAFC, identical or nearly identical numbers of cells are detected in the case of each of the treated and untreated cultures for homogeneous resistant strains. In contrast, heterogeneous resistant strains were those strains in which a slight retardation in growth was observed as illustrated in FIGS. 4C and 4D. Many genotypically resistant strains displayed an intermediary growth in the presence of daptomycin. The level of growth inhibition is correlated to the amount of penetrance of mecA gene expression in that population. Heterogenous resistant strains A and B (FIGS. 4B and 4C, respectively) are representative examples of strains in which growth curves showed an intermediary inhibition (that is, intermediate to the inhibition of homogeneous susceptible and homogeneous resistant strains) in the presence of daptomycin. When heterogeneous resistant strains are tested in the PAFC system of FIG. 3, corroboration is observed with the growth curves and the intermediary number of cells detected. Heterogeneous Resistant B in FIG. 4D demonstrated both a delay in growth and large, but not complete, inhibition of growth.

Combining the growth curve data and PAFC results lead to the determination that Heterogenous A and B strains are heterogeneous in their expression for the mecA gene. The results indicate that strain Heterogeneous Resistant A in FIG. 4C expresses resistance derived from the mecA gene at a higher penetrance while Heterogeneous Resistant B in FIG. 4D expresses the mecA gene at a low penetrance. RNASeq may be performed to further correlate the level of gene expression penetrance with growth curves and cells detected from PAFC.

The devices, systems and methods hereof exploit the specificity of, for example, naturally derived bacteriophage probes and the robust nature of laser induced ultrasonics to provide a rapid, unambiguous method for objective identification of bacterial species and their antibiotic susceptibility in a wide variety of sample media. The results of studies hereof demonstrate, for example, the ability of the devices, systems, and methods hereof to identify antibiotic resistance in Staph. aureus in less than 4 hours. This 4-hour period includes 2 hours of incubation with and without antibiotic, followed by photoacoustic testing, which is 2 hours or less. Additionally, the results of the studies hereof show an ability to identify heterogeneous resistant strains that are often misidentified. In addition to advantages in patient treatment, correct identification of heterogeneous resistant Staph. aureus could, for example, potentially save hospitals money and resources.

Thresholds for characterization or classification of bacteria as homogeneous resistant, heterogeneous resistant, or susceptible to a particular antibiotic treatment may be developed in the methodologies hereof. Such thresholds may, for example, be context sensitive. Further, a clinician can readily distinguish between such strains of bacteria in practicing the methods hereof as illustrated in the representative studies set forth in FIGS. 4A through 4C. Moreover, one or more computer implemented artificial intelligence (for example, machine learning) algorithms may be used in characterizing the results hereof.

Referring again to FIG. 3, an embodiment of a system 10 hereof includes a photoacoustic flow cytometry system or PAFC system 100 in operative connection with electronic circuitry 200 which may, for example, be used for control of PAFC system 100 and/or processing/analyze data therefrom. In the illustrated embodiment, electronic circuitry 200 includes a processor system and a memory system in communicative connection with the processor system. The processor system and memory system may, for example, be embodied in a single computer or be distributed over multiple devices and/or locations. A database system may, for example, be stored in the memory system. The database system may, for example, include data associated with previous characterization of bacteria strains from PAFC system 100 and/or other PAFC systems. The data of the database system can be continuously updated or enhanced with data from PAFC system 100 and/or other PAFC systems (for example, via a cloud-based system). Such data may, for example, be used to train one or more machine learning models of one or more algorithms stored in the memory system and executable by the processor system. The machine learning algorithm(s) used in embodiments hereof may be unsupervised or supervised/trained (for example, on the basis of known data and expert therapist/clinician characterization) using collected data from PAFC system 100 and/or other PAFC systems.

Characterization algorithm(s) hereof may, for example, include one or multiple machine learning algorithms to obtain better characterization performance. Representative examples of individual machine learning algorithms suitable for use herein include, but are not limited to, clustering algorithms, classification algorithms, and/or regression algorithms. In a number of embodiments, one or more individually employed machine learning algorithms or models may be incorporated or combined into one single machine learning algorithm and model. As known in the artificial intelligence arts, a machine learning model is the output generated when “machine learning algorithm” is trained with a training data set. In a number of embodiments including a combination of machine learning algorithms/models, the combined algorithm/model may output characterizations for each of the individual algorithms and may selects the model prediction with the best outcome best confidence interval. Machine learning algorithms/models hereof may be evaluated against a prior determined test data set on a set of evaluation metrics including, but not limited to, accuracy, precision, learning performance, and prediction performance.

In a representative example, k-means clustering was used in characterizing Staphylococcus aureus or S. aureus as resistant or susceptible to the antibiotic oxacillin with 100% accuracy. A discussion of k-means clustering is provided at Arthur, D. and S. Vassilvitskii. k-means++:the advantages of careful seeding. in SODA '07. 2007, the disclosure of which is incorporated herein by reference. Although a clinician may interpret the number of bacteria after oxacillin treatment compared to the number in the untreated subsample as described above to readily determine whether the bacteria is homogeneous susceptible, homogeneous resistant, or heterogeneous resistant, a formal/objective method to automatically determine resistance may provide advantage. K-means, like other clustering methods, takes data points in a space of one or more dimensions and determines natural groupings of those points by proximity. Given a number of clusters, k, the algorithm separates all points into that number of groups. K-means clustering may thus provide an objective means for separating the set of samples into groups. Although k-means clustering provides consistent results with prior determined nature of samples, classifiers, rather than a clustering methods, may be developed to improve results. As clear to those skilled in the computer arts, given suitable training data sets, supervised classification algorithms may readily be developed/trained for rapid characterization of results in the methodologies hereof. Machine learning algorithms such a classification algorithms may, for example, be useful in characterizing a large number of studies.

In a representative study, total of thirteen samples were tested for the mecA gene using PCR, thus determining their methicillin resistance before photoacoustic testing. Streaks of each S. aureus strain were grown on mannitol salt agar plates. Single colonies from each streak plate were used to regrow strains in mannitol salt broth for 2 hours in a shaking water bath at 36.5 C. That period ensured cells were growing and entering exponential growth phase. Oxacillin was added at a final concentration of 1 μmg/ml to half of each culture and grown for an additional 2 hours. Before processing through PAFC system, 100 μl from each culture was removed and used for growth analysis in an HI plate reader (available from Biotek of Winooski, Vermont). Growth curves were made for each culture by taking the optical density of each treated and untreated culture every minute over a 16-hour period. As described above, it was determined that two hours of antibiotic treatment was sufficient to determine differential growth rates from prior experimentation. Prior to performing photoacoustic testing, treated and untreated samples were incubated side-by-side for two hours. Photoacoustic testing of treated and untreated samples for each isolate were alternated, so that both samples were tested within twenty minutes to allow for similar growth times. Thus, total bacteria number could be compared properly.

TABLE 1

| | | | | mecA | Concordant |
|---|---|---|---|---|---|
| | Untreated | Treated | | verified | for k-means |
| Isolate | Detections | Detections | k-means | by PCR | and PCR |
| K3255 | 798 | 53 | 1 | No | Yes |
| K3251 | 726 | 7 | 1 | No | Yes |
| B1899 | 611 | 404 | 2 | Yes | Yes |
| K3282 | 818 | 689 | 2 | Yes | Yes |
| K3266 | 119 | 18 | 1 | No | Yes |
| K3268 | 88 | 110 | 2 | Yes | Yes |
| K3270 | 144 | 134 | 2 | Yes | Yes |
| K3279 | 198 | 175 | 2 | Yes | Yes |
| K3261 | 170 | 135 | 2 | Yes | Yes |
| K3262 | 183 | 10 | 1 | No | Yes |
| K3237 | 137 | 44 | 1 | No | Yes |
| K3287 | 210 | 2 | 1 | No | Yes |
| K3280 | 227 | 179 | 2 | Yes | Yes |

To interpret the photoacoustic data provided by the flow cytometer, k-means clustering was used to guide the differentiation between methicillin resistant and susceptible samples. Although a simple interpretation of the number of bacteria after oxacillin treatment compared to the number in the untreated subsample may indicate whether the bacteria was methicillin resistant or not, a formal method was used to demonstrate ready automation of determining resistance. K-means, like other clustering methods, takes data points in a space of one or more dimensions and determines natural groupings of those points by proximity. Given a number of clusters, k, the algorithm separates all points into that number of groups. In a representative study, there was an interest in identifying only resistant and susceptible groups. Thus, k=2 was chosen.

The ratio of treated detection numbers over untreated numbers was determined, resulting in 13 numbers which were approximately in the range of 0 to 1. Lower numbers indicate that oxacillin was effective in decreasing the S. aureus population. However, there was no ad hoc threshold for determining antibiotic resistance. K-means with two clusters was applied to the data set using the MATLAB programming language (available from Math Works of Natick, Massachusetts). The MATLAB function, kmeans, uses Lloyd's algorithm. For simplicity, Euclidean distance was used for measuring and establishing iterative clusters. The analysis resulted in two clearly defined clusters for MRSA and methicillin susceptible samples.

Table 1 lists each of the clinical isolates obtained from the patients of the study. Table 1 indicates how many photoacoustic events and, hence, how many bacterial cells, were detected in the oxacillin-treated and untreated subsamples. Photoacoustic testing resulted in bacterial counts ranging from 2 to 689 at 2 hours when incubated with oxacillin compared to 88 to 818 when samples incubated without antibiotics. Because the sample size was relatively small and the distribution was not obviously Gaussian, a nonparametric test was performed to compare the means of the bacterial counts before and after treatment with oxacillin. Using a Wilcoxon matched pairs signed rank test, a p-value of 0.0007 was calculated.

Two distinct subpopulations were observed after incubation with oxacillin. In one subgroup, growth rates were similar between treated and untreated conditions, with a mean ratio of treated to untreated of 0.87, while the second group was markedly different with a mean ratio of 0.10. Once again, due to the limited sample size, a nonparametric test of the two groups was performed. Using a Mann-Whitney test, a p-value of 0.0012 was calculated. Seven of the 13 clinical isolates were found to be methicillin resistant using PCR testing for mecA gene. Isolates with the mecA gene corresponded to the subgroup with similar growth rates.

To confirm the groupings by photoacoustic detection results, an unbiased clustering method, k-means was used. The k-means column in Table 1 shows whether the numbers determined a ratio that clustered in group 1 or 2, as determined by the MATLAB algorithm. The k-means algorithm resulted in 100% concordance with the known antibiotic resistance using mecA genetic analysis. Although k-means clustering was consistent with the prior determined nature of the samples, a classifier may, for example, be used, rather than a clustering method, so that one can determine resistance, susceptible, or heterogeneous resistance to antibiotics from single samples in the clinic.

S. aureus is a common cause of bacterial keratitis, conjunctivitis, and endophthalmitis. The samples used in the above-described study were obtained from clinical cases of keratitis. While vancomycin is often used for treatment of MRSA keratitis, it is associated with corneal toxicity. The clinical significance cannot be understated, as MRSA keratitis is often part of a series of comorbidities that affect visual function. While photoacoustics can certainly identify the foundational bacterial infection and provide insight into factors that can be used to manage therapy for the infection, the photoacoustic method may be adapted for wider application in keratitis, which manifests in a complex environment that is still clinically challenging.

Although a number of representative studies hereof used daptomycin and oxacillin as representative examples of an antibiotic and S. aureus as a representative bacteria, one skilled in the art appreciates that the embodiments of PAFC-based devices, systems, and methods hereof may be extended to determine antibiotic sensitivity to other types of antibiotics, bacteria, and resistance. Without limitation, exemplary classes of representative antibiotics include aminoglycosides, carbapenems, cephalosporins, quinolones/fluoroquinolones, glycopeptides and lipoglycopeptides (such as vancomycin), macrolides (such as erythromycin and azithromycin), monobactams (aztreonam), oxazolidinones (such as linezolid and tedizolid), penicillins, polypeptides, rifamycins, sulfonamides, streptogramins (such as quinupristin and dalfopristin), and tetracyclines. The carbapenems, cephalosporins, monobactams, and penicillins are subclasses of beta-lactam antibiotics, a class of antibiotic characterized by a chemical structure called a beta-lactam ring. Further representative antibiotics that do not fit into the classes listed above include chloramphenicol, clindamycin, daptomycin, fosfomycin, lefamulin, metronidazole, mupirocin, nitrofurantoin, and tigecycline. The methodologies hereof can be conducted with single antibiotics or mixtures thereof. Additionally, RNA-Seq may be performed to further correlate the level of gene expression penetrance with growth curves and cells detected from PAFC.

Vancomycin resistant strains, though less common, are dramatically harder to treat and use up hospitals limited resources. Vancomycin-resistant Enterococcus, carbapenem-resistant Enterobacteriaceae, and multi-drug-resistant Pseudomonas aeruginosa are listed as serious threats by the CDC and would benefit from early detection and susceptibility determination. On both an individual and global scale, rapid identification and characterization is essential. The potential for worldwide crippling pandemics from bacterial pathogens is of central concern to the CDC and WHO. Multi-drug-resistant bacterial infections regularly have mortality rates closer to 50% and transmission rates similar to that of flu and SARS-COV-19 which have mortality rates closer to 1%. There is a clear need for better and more advanced rapid diagnostics to protect individual patients. The disk diffusion method has been the gold standard since 1956 but requires 16-24 hours after cultures are grown. PAFC has the potential to supplant the gold standard by directly counting the difference between cells in treated and untreated cultures in less than four hours.

Production and purification of phage may be a limiting factor for the number of samples that can be tested in, for example, a laboratory setting. Although the production and purification of bacteriophage is relatively inexpensive, even on a laboratory scale it is time consuming and requires specialized skills. However, with advancements in phage therapy, several companies such as Advanced Phage Therapeutics (Gaithersburg, MD) and ARMATA pharmaceuticals (Marina del Ray, CA) have started large scale production of FDA approved Good Manufacturing Practice or GMP phage. The availability of high titer purified phage significantly increases the practicality and lowers the expense of the devices, systems, and methods hereof for bacterial detection. Additionally, the devices, systems, and methods hereof may also be used in phage therapy to rapidly test bacterial susceptibility to particular phage for treatment purposes.

Photoacoustic flow cytometers are relatively economical to build and are much simpler than many common laboratory equipment. Laser sources are the components with the greatest cost, and all parts are commercially available. Total cost of a laboratory setup is around $30,000 while commercial setups could be produced for much less. Such low equipment cost indicates photoacoustic flow cytometry could become a common clinical tool, similar to x-ray or ultrasound machines.

The methodologies hereof and PAFC-based devices, systems, and methods using labeled bacteriophage generally may be further improved by using labeled bacteriophage in which the lysing process of the bacteriophage is delayed or inactivated. Delaying/inactivating the lysing process of bacteriophage may, for example, be accomplished by removing a portion of all of the nucleic acid of the bacteriophage while retaining the activity to selectively bind to bacteria in the remaining portion of the bacteriophage. For example, in bacteriophage including a capsid and tail, one may remove the capsid or a portion thereof to provide a labeled tail as illustrated in FIG. 2 which retains the ability to selective bind to a species of bacterial but lacks the nucleic acid required to initiate lysing. Alternatively, the nucleic acid can be removed from a bacteriophage while retaining the capsid (for example, by making it porous to remove nucleic acid). Retaining the capsid while removing the nucleic acid therefrom may enable increased loading of dye within the interior volume of the capsid as illustrated in FIG. 2. A bacteriophage or phage virion that lacks nucleic acid is referred to as a bacteriophage/phage ghost, a ghost particle, or ghost. Nucleic acids can be removed from bacteriophage via a number of methodologies, for example, via osmotic shock etc. Bacteriophage ghosts can retain their adsorption competence. The use of portions of bacteriophage such as ghost particles, bacteriophage sections (for example, tails or a section thereof wherein the capsid has been partially or fully removed), or a bacteriophage host-recognition proteins (or a synthetic host-recognition proteins based on bacteriophage host-recognition proteins) may prevent bacterial lysis in patient samples being prepared for photoacoustic flow cytometry because of the lack nucleic acid, thereby allowing for longer timescales for testing. Delay or cessation/inactivation of the lysis process may, for example, reduce operator dependence.

The labeling agents used herein may be extending to entities other than bacteriophages and portions of bacteriophage (for example, bacteriophage tails) using labeling chemistry similar to that developed for bacteriophage. As set forth above, labeled antibodies may be used as labeling agents herein. Moreover, various proteins (for example, antibodies or host-recognition proteins), peptides, and/or other entities that selective bind to bacteria (for example, via receptors thereon) may also be conjugated with photoacoustic responsive labels or tags and be used as bacterial labeling agents in the devices, systems, and methods hereof. For example, bacteriocins are proteinaceous or peptidic toxins that are produced by bacteria to inhibit the growth of similar or closely related bacterial strain(s) and may be used as labeling agents hereof. Pyocins are, for example, a subset of bacteriocins that may be used as labeling agents herein. Pyocins are produced by more than 90% of Pseudomonas aeruginosa strains, and each strain may synthesize several pyocins. The pyocin genes are located on the P. aeruginosa chromosome and their activities are inducible by mutagenic agents such as mitomycin C. Three types of pyocins are described. (i). R-type pyocins resemble non-flexible and contractile tails of bacteriophages. They provoke a depolarization of the cytoplasmic membrane in relation with pore formation. (ii). F-type pyocins also resemble phage tails, but with a flexible and non-contractile rod-like structure. (iii). S-type pyocins are colicin-like, protease-sensitive proteins. They are constituted of two components. The large component carries the killing activity (DNase activity for pyocins S1, S2, S3, AP41; tRNase for pyocin S4; channel-forming activity for pyocin S5). It interacts with the small component (immunity protein).

As set forth above, R-type pyocins resemble inflexible and contractile tails of bacteriophages and are further classified into five groups: R1, R2, R3, R4, and R5. They are similar to each other in their structural and serological properties, but they are different in receptor specificity. The tail fiber protein, an apparatus for binding to the receptor of a sensitive bacterial strain, has been proposed to account for the main difference. The receptors for R-type pyocins are lipopolysaccharides or lipooligosaccharides found in the outer membrane. R-type pyocins, when used to challenge sensitive cells, provoke a depolarization of the cytoplasmic membrane in relation to pore formation and inhibit active transport. Contraction of the tail-like structure is necessary for this bactericidal action.

In another methodology for determining antibiotic resistance hereof, one need not separate a sample into a plurality of subsamples to determine responses of an antibiotic-treated subset and an untreated subset. In that method, a determination is first made that a particular species, strain, or type of virus is present in a sample using, for example, the devices, methods, and systems disclosed in U.S. Pat. Nos. 10,544,443 and 10,961,557. In that regard, in a representative example of Staph aureus bacteria, one may, for example, use a broad spectrum labeled bacteriophage, such as SP1, which infects 98% or more of Staph aureus bacteria as a speciating labeling agent to determine the presence of Staph aureus bacteria using a photoacoustic detection system. Once a patient sample has been identified as positive for Staph aureus using, for example, a labeled SP1 bacteriophage, the sample may be subjected to a second Staph phage that specifically targets bacteria that are resistant to antibiotics. For example, the BI bacteriophage for Staph aureus targets methicillin resistant strains thereof. The bacteriophage that targets resistant bacterial may be labeled or tagged with a label that is detectible via a photoacoustic detection system or detection system including a photoacoustic cell. Such a labeled bacteriophage is added to a patient sample and the sample is subsequently characterized via a photoacoustic detection system to determine the presence of resistant bacteria strains.

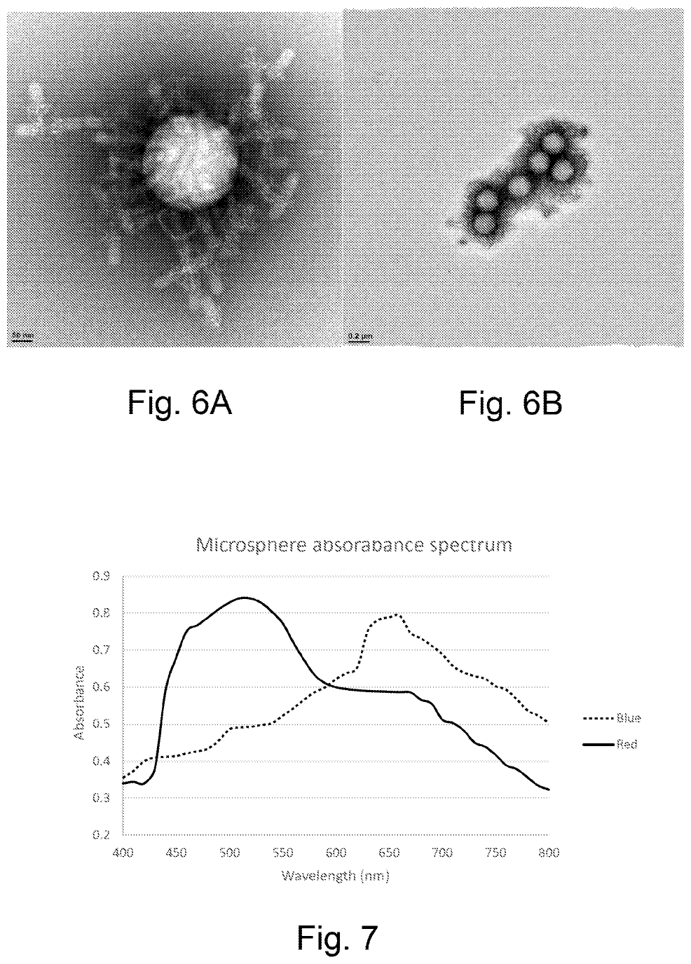

In another embodiment hereof, functionalized solid particles were used in connection with an entity which selectively binds to a species of bacteria. Nanoparticles or microparticles for use herein may, for example, be solid particles between 1 nm and 500 μm in size, 10 nm and $00 μm in size, or 10 nm and 100 μm in size. In a number of embodiments, such particles are metal particles (for example, gold, silver, iron, etc.), polymeric polymers (for example, polystyrene, etc.), ceramics, or particles of naturally occurring materials (for example, minerals, melanin, etc.). In a number of embodiments including in which the particle is not naturally or inherently absorbing/detectible via a photoacoustic system (for example, in the case of certain polymeric particles), compounds detectible via a photoacoustic detection system are added to the particles. In the case of a number of particles of synthetic or naturally occurring materials (for example, particles of melanin), the particles may be naturally or inherently detectible via a photoacoustic detection system. In general, in the case of particles which do not inherently absorb light energy and emit acoustic waves, compounds which are detectible via a photoacoustic detection system may be added thereto. In a number of embodiments, generally spherical, dye-containing particles or spheres (for example, polymeric nanospheres or microspheres) were used as representative examples. In a number of representative examples, particles including photo-absorbing functionality were, for example, attached to one or more entities which selectively binds to the bacteria (for example, bacteriophage, bacteriophage ghosts, tails of a bacteriophage from which at least a portion of a capsid thereof had been removed, etc.) in a manner that the entities remain functional to selectively bind to bacteria. In a number of studies, functionalized microspheres were produced by, for example, attaching bacteriophage tails directly to a biotin-binding-protein-coated (for example, streptavidin-coated), colored photo-absorbing microspheres. Bacteriophage tails contain the host attachment proteins responsible for selectivity and specificity in bacteriophage binding. FIGS. 6A and 6B illustrate, respectively, a single functionalized microsphere with multiple bound phage tails in random orientations and multiple functionalized microspheres with attached phage tails. Attachment mechanisms other than avidin-biotin interactions may be used to attach an entity which selectively binds to bacteria to a particle such as, but not limited to, covalent bonding, ionic bonding, charge-charge interaction, and other protein-protein interactions. In general, other attachment/interaction pairings such as protein-protein or protein-small molecule pairings with relatively tight-binding properties or small dissociation constants, similar to the biotin-avidin interaction, are readily adapted for user herein.

Electron micrographs such as those set forth in FIGS. 6A and 6B confirmed complete coverage by bacteriophage tails to the surface of each microsphere. Bacteriophage tails were randomly oriented on the microsphere surface as confirmed by electron microscopy. It has been shown that randomly oriented phage tails bind at nearly the same efficiency as oriented phage tails. Therefore, significant reduction in binding capacity is not expected from the random orientation of tails as each microsphere has multiple tails attached allowing for multiple attachment points. While confirming the tail orientation, a slight overabundance of tails was noticed. To optimize, microspheres may be produced with a lesser number of attached tails to maximize the use of purified tails and minimize overcrowding issues.

By attaching bacteriophage tails to commercially available particles such as microspheres one may leverage the high signal produced from microspheres with the attachment and irreversible binding provided by bacteriophage tails. The uniform size and color of, for example, polymeric microspheres allows for a uniformity of signal. Uniform signal size allows one to accurately determine peak thresholds for detections. Additionally, the robust and consistent signal obtained from such microspheres allows one to increase our signal to noise ratio. Additionally, use of bacteriophage portion from which DNA has been eliminated while retaining selective binding through the presence of one or more host-recognition proteins allows one to remove the potential for lysis of bacterial cells as described above. The inhibition of lysis enables the downstream capture and testing of detected bacterial cells. Captured cells can then be sequenced or potentially clonal colonies could be grown from them to further analysis.

Control experiments, which are summarized in Table 2 below, were conducted which demonstrated positive signals from black microspheres while obtaining zero signals from PBS, target and non-target bacteria, and red- and blue-functionalize microspheres.

TABLE 2

| Control Type | Sample | Detections |
|---|---|---|
| Positive | Black 1 μM Microsphere | 967 |
| Negative | PBS | 0 |
| Negative | LT2 bacteria alone | 0 |
| Negative | K12 bacteria alone | 0 |
| Negative | 1X Red streptavidin 0.2 μM | 0 |
| | Microsphere | |
| Negative | 1X Blue streptavidin 0.2 μM | 0 |
| | Microsphere | |
| Negative | 10x Red streptavidin 0.2 μM | 0 |
| | Microsphere | |
| Negative | 10x Blue streptavidin 0.2 μM | 0 |
| | Microsphere | |

To test the binding and detection of the functionalized microsphere each combination of target and non-target bacteria was tested with red- and blue-functionalized microspheres. Bacterial strains were grown, and cultures diluted to roughly 100 cells per test. Microspheres were added to approximately 500 functionalized microsphere per bacterial cell or 50,000 per test. The results are listed in Table 3 below.

TABLE 3

| Microsphere | Bacterial strain | Trial #1 | Trial #2 | Trial #3 |
|---|---|---|---|---|
| RFM | LT2 | 115 | 114 | 99 |
| RFM | K12 | 3 | 3 | 0 |
| BFM | LT2 | 75 | 84 | 101 |
| BFM | K12 | 0 | 0 | 1 |

To demonstrate a similar level of detection between red-functionalized microspheres and blue-functionalized microspheres hereof a Kruskal-Walls statistical test was performed. The goal of this analysis is to demonstrate that both red- and blue-functionalized microspheres discriminate the same. The results of this test show that the medians are unequal. Therefore, both functionalized microspheres discriminate similarly. Any differences in detections can be assumed to be due to absorbance. To test this the absorbance spectrum of each colored microsphere were determined, and the results are shown in FIG. 7.

In a number of studies hereof in which two colors of microspheres and binding to two different bacterial hosts were studied, tails from bacteriophage Det7 were used to produce both red- and blue-functionalized microspheres. Det7 is a bacteriophage that exclusively binds to Salmonella bacterial species. As a non-target host E. coli K12 was use. Det7 does not bind to or infect E. coli K12. Red- and blue-functionalized microspheres were tested in triplicate in PAFC system 100 hereof with Salmonella strain LT2. Red microspheres showed roughly 20% more detections with an average number of detections of 109.3 for red and 86.7 for blue microspheres. There are several possible reasons for this discrepancy in number of detections. First, the red microspheres absorb the green laser light slightly better than the blue microspheres of the same size. It is likely that the difference in absorbance accounts for the roughly 20% difference in detections. It is also possible, though unlikely, that the dilutions of each bacterial culture were slightly skewed. Despite these differences in detections for target bacteria, the number of detections for non-target bacteria are consistently zero indicating very good discrimination between bacterial strains and specificity of binding.

Combining the host attachment specificity of bacteriophage host-recognition proteins as, for example, present in bacteriophage tails and the uniform production and absorption of dyed microspheres such as polystyrene microspheres allows one to quantitate specific bacterial contaminants. This technique allows for producing multi-target microspheres with any combination of binding produced by bacteriophage. The binding of bacteriophage host-recognition proteins is often to essential cell surface proteins making them far superior to antibody detection. Moreover, as described above, phage attachment/host-recognition proteins have been shown to be some of the most stable protein structures discovered, allowing ease of storage and long-term viability of functionalized microspheres as bacterial probes.

Photoacoustic flow cytometry in conjunction with functionalized particles such as nanospheres or microspheres presents a method of rapid bacterial detection and quantification with the added benefits of uniform signals and potential recovery of each detected bacterial cell. Further development of this methods and combining this technique with method of determining antibiotic sensitivities shows the potential for clinical applications and point of care use. Rapid detection and identification of bacterial infection are not only a cost saving, but also, and more importantly, a potential lifesaving technology. Frequently, the limiting factors for patient treatment is the time spent waiting for results.

Multi-host functionalized microspheres may be produced as described herein with wide target host ranges allowing for even more rapid identification of bacterial contamination. Bacteriophage host range differs dramatically between types of bacteria. Salmonella, for example, has over 2600 serovars, all with different antigens for bacteriophage attachment. Very few bacteriophage have broad host ranges of Salmonella. Staph aureus has several bacteriophage with extremely broad host range due to the similarity of binding sites of the teichoic acids.

In a number of representative embodiments, multi-host particles may, for example, be focused on pathogenic versus non-pathogenic enteric bacteria. The ability to rapidly differentiate between non-pathogenic E. coli strains and pathogenic E. coli such as o157:H7 will be very valuable to, for example, the food industry. Real-time monitoring of food processing facilities or water purification facilities for pathogenic enteric bacteria may, for example, have substantial impacts, particularly in third-world countries. Another target may, for example, be opportunistic blood-borne pathogens such as ESKAPE pathogens. ESKAPE is an acronym formed from the scientific names of six very virulent and antibiotic-resistant bacterial pathogens as follows: Enterococcus faecium, Staphylococcus aureus, Klebsiella pneumoniae, Acinetobacter baumannii, Pseudomonas aeruginosa, and Enterobacter spp. Rapid identification of the presence of any of the ESKAPE pathogens allows for further testing to identify a specific one and subsequently initiate rapid antibiotic sensitivity testing.

### Experimental Examples

### Materials and Methods.

The photoacoustic setup (see FIG. 3) is similar to that described in U.S. Pat. Nos. 10,544,443 and 10,961,557. PAFC has been described previously and was shown to be a robust method of detecting rare particles in dilute samples. The PAFC system was tested with phosphate buffered saline (PBS) to demonstrate a level of background noise or variability. As a positive control for system function, we used 10 μm polystyrene spheres obtained from Polybead (Warrington, PA) and titered them through the PAFC system. Staph. aureus strain SA113 (ATCC 35556, Manassas, Virginia) was obtained from American Type Culture Collection and we used E. coli K12 as a control bacterial cell type. Dyed SP1 bacteriophage were added to resuspended cultures at a ratio of 1000 phage per bacterial cell. Phage/bacteria mixtures were incubated on the bench top for 10 minutes to allow phage attachment, then processed thought PAFC system.

Sample preparation. Clinical isolates of Staph. aureus were obtained from the Urisch laboratory at the UPMC Department of Othopaedic Surgery. Isolates were de-identified according to IRB protocol and were stored in 50% glycerol solution at −80° C. Isolates were streaked for single colonies on mannitol salt agar (MSA) plates and cultures were grown in mannitol salt broth, shaking at 36.5° C. Overnight cultures were then diluted into fresh media and regrown for 2 hours to ensure that bacteria were entering exponential growth phase. Exponentially growing cultures were mixed with media or daptomycin in a 1:1 ratio. Daptomycin was added to 0.5 ml of culture to a final concentration of 0.25 μg/ml. Identical cultures were used in all experiments using PAFC and BioTek HI plate reader. For photoacoustic testing, bacteriophage SP1 was added to a concentration of 1000 phage per bacterial cell. Growth curves were performed for 16 hours with measurements taken once every minute. Growth curves for each strain and corresponding PAFC results are displayed in FIG. 4A through 4D.

Treated and control cultures were placed in a round bottom 96 well culture plate (Falcon microtest 96 well plate 35077, ThermoFisher, Waltham, Massachusetts) and were placed into the BioTek HI plate reader. Optical density (OD) measurements were taken every minute at 600 nm wavelength. Between measurements, the plate was shaken at 100 rpm and maintained at 36.5+C allowing for bacterial growth. The two-hour time point was determined to be sufficient to differentiate the growth rates. Additionally, multiple replicates of each bacterial strain were grown at 36.5° C. in the BioTek HI plate reader and 100 μL was removed and plated on MSA plates every 10 minutes. Growth curves were made directly from these titers for each strain to give a quantifiable number of bacterial cells for each OD. Each strain was found to consistently correlated between cell titer and OD.

Photoacoustic Flow Cytometry. The photoacoustic flow chamber is shown in FIG. 3. A 1000 μm optical fiber with numerical aperture of 0.39 (Thorlabs, Newton, New Jersey) was used to deliver laser light at 532 nm with a 5 ns pulse duration from a Nd:YAG laser (Litron Nano, Boseman, Montana). Laser beam energy was maintained and measured between 1.9 and 2.1/mJ for all PAFC experiments. The optical fiber was placed 5 mm away from a quartz tube (Quartz 10 QZ, Charles Supper, Natick, Massachusetts) with 10 μm thick walls. Samples were pumped through the quartz tube inside a 3D printed flow chamber. The laser beam was assumed Gaussian in shape and fluence was calculated to be 0.014 mJ/cm² and detection volume was determined to be 0.04 μL.

Sonotech LithoClear acoustic gel (Next Medical Products Company, Branchburg, New Jersey) filled the internal space of the 3D printed flow chamber, and a 2.25 MHz transducer was focused on the quartz sample tube. The acoustic gel provided acoustic coupling between the quartz tube and the transducer along the propagation of acoustic waves generated from thermoelastic expansion. A Tegam 4040B amplifier (Tegam, Inc., Geneva, Ohio) amplified signals with a gain of 50. A computer running a LabView program recorded signal waveforms.

To allow for microfluidic capture of detected cells, in a number of embodiments, two phase flow was introduced. An immiscible liquid, mineral oil, was introduced into our sample flow to create alternating droplets of oil and sample. These alternating droplets ensured cells could not be retained in the flow system and allowed for localized detection and isolation of acoustic events. The detection of acoustic events was accomplished using the focused transducer coupled to an amplifier (National Instruments, Austin, Texas) connected to a desktop computer (Dell, Round Rock, Texas) running our LabVIEW (National Instruments, Austin, Texas) program built for recording and quantifying acoustic events.

To determine background noise the photoacoustic flow cytometry (PAFC) system was tested using phosphate buffered saline (PBS) and Phage Buffer (10 mM Tris, pH 7.5, 10 mM MgCl₂, 68 mM NaCl). As a positive system control, 10 μm polystyrene microsphere (Polybead, Warrington, PA) were tested and tittered through the PAFC system. Bacterial cultures of LT2 Salmonella and K12 E. coli were diluted into PBS and tittered through the PAFC system. Additionally, 0.2 μm streptavidin coated dyed microspheres were tittered through the system and concentration of microspheres was increased until microspheres were able to be detected.

Bacteriophage preparation. SP1 bacteriophage were grown using Staph. aureus strain SA113 (ATCC, Old Town Manassas, Virginia) and concentrated using methods described previously. Purified phage of 1×10¹² plaque forming units per milliliter (PFU/ml) were added to a saturated solution of Direct Red 81 dye (Sigma Aldrich, Saint Louis, Missouri), Virion particles were then pelleted and resuspended in buffer (10 mM Tris, pH 7.5, 10 mM MgCl2, 68 mM NaCl). This process was repeated to ensure the removal of unbound dye. The absorbance spectrum of dyed phage was determined using the BioTek HI and compared to that of undyed phage particles. Dyed phage were titered to ensure no detrimental effects were observed from the dying process. Dyed phage were retested for their ability to infect after 150 days and no difference in titer was observed.

All strains were tested in the presence and absence of daptomycin. Cultures were diluted into fresh media and regrown for 2 hours in the presence and absence of 25 μg/ml of daptomycin. Each sample was then incubated at room temperature for 10 minutes with multiple dyed phage per bacterial cell. Incubated samples were then processed through the PAFC system and number of detected cells recorded. Isolates were tested in triplicate for both the plate reader and PAFC system.

Ghost bacteriophage preparation. Ghost particles can be prepared using a number of procedures. In one embodiment, 10 mL of high concentration Det7 bacteriophage is obtained, and the phage is transferred into a piece of dialysis tubing having a length such that each end is secured well. 90 mL of 2.3 M sodium perchlorate (NaClO₄) is transferred into a beaker large enough to hold the solution and the dialysis tubing from the previous step. For 2.3M: 90 mL of DI water and 25.35 g of NaClO₄ are combined and stirred until dissolved. 100 microliters of 50 mM EDTA is added to this solution The tubing should remain submerged in the solution for 30 minutes. The tubing is then removed from the solution and immediately place in a beaker of Phage Buffer, which stops the reaction. The DNA will begin to coagulate in the tube and, to eliminate it, DNAse is added to the tubing to remove excess DNA

Bacteriophage dying protocol. Ethidium Bromide (sometimes referred to herein as EthBr), Propidium Iodide (sometimes referred to herein as ProId), Direct Red (sometimes referred to herein as DRED), DRED and EthBr, DRED and ProId dyes are used in the protocol. The following protocol is completed for each of the above-identified dyes:
1. Complete the following protocol for above dyes:
   a. Place 100 microliters of Det7 at 10¹² pfu/ml in a 250 microliter PCR tubes along with 100 microliters of dye.
      i. If two dyes are being used for this use a PCR tube: 100 microliters of Det7 at 10¹² pfu/ml, 50 microliters of first dye and 50 microliters of second dye. The final volume will be 200 microliters.
      ii. If three dyes are being used for this use a PCR tube: 100 microliters of Det7 at 10¹² pfu/ml, 50 microliters of first dye, 50 microliters of second dye, and 50 microliters of third dye. Final volume of 250 microliters. (There is a decrease in dye concentration for this step, but there is no effect since the dye is still in excess.)
   b. Control, 100 microliters of Det7 at 10¹² pfu/ml and 100 microliters of phage buffer.
   c. Heat in the thermocycler for one hour at 50 degrees C.
   d. Once heating is complete, transfer contents of PCR tube to Eppendorff tube that has 800 microliters of phage buffer. Resulting volume of 1 mL (10¹¹ pfu/ml)
      i. For tubes with two dyes, transfer the contents of both PCR tubes from step i. into an Eppendorff tube containing 800 microliters of phage buffer. Resulting volume of 1 mL.
      ii. For tubes with three dyes, transfer the contents of both PCR tubes from step i. into an Eppendorff tube containing 750 microliters of phage buffer. Resulting volume of 1 mL
   e. Centrifuge at 14,000 rpm for 3 hours at 20 degrees C.
   f. Remove supernatant, wash pellet with 1 ml of phage buffer Ix, then resuspend pellet with 1 mL of phage buffer by vortexing and leave in the fridge overnight.
      i. Final phage titer (10¹¹ pfu/ml)
2. Complete the following protocol for both dyes:
   a. Place 100 microliters of Det7 at 10¹² pfu/ml in PCR tubes along with 100 microliters of dye.
      i. If two dyes are being used for this use a PCR tube: 100 microliters of Det7 at 10¹², 50 microliters of first dye and 50 microliters of second dye. Final volume of 200 microliters.
      ii. If three dyes are being used for this use a PCR tube: 100 microliters of Det7 at 10¹² pfu/ml, 50 microliters of first dye, 50 microliters of second dye, and 50 microliters of third dye. Final volume of 250 microliters. (There is a decrease in dye concentration for this step, but there is no effect since the dye is still in excess.)
   b. Control, 100 microliters of Det7 at 10¹¹ pfu/ml and 100 microliters of phage buffer should be prepared.
   c. Leave both tubes on the bench top for 3 hours.
   d. Once incubation is complete, transfer contents of PCR tube to Eppendorff tube that has 800 microliters of phage buffer. Resulting volume of 1 mL.
      i. For tubes with two dyes, transfer the contents of both PCR tubes from step a.i. into an Eppendorff tube containing 750 microliters of phage buffer. Resulting volume of 1 mL.
   e. Centrifuge at 14,000 rpm for 3 hours at 20 degrees C.
   f. Remove supernatant, wash pellet with 1 ml phage buffer, then resuspend pellet with 1 mL phage buffer in the fridge overnight.

### Controls

1. Undyed phage: The tube is a clear liquid, after spinning there is a large hazy pellet in the very bottom of the tube.
2. Undyed ghost: The tube is a clear liquid, after spinning there is no visible pellet.
3. Dyed phage w/DirectRed81: The liquid is bright red and the pellet after spinning is large and has an orange tint.
4. Phage w/Ethidium Bromide: the liquid is bright red, and the pellet resembles that of the previous tube, reddish orange pellet.
5. Phage w/Propidium Iodide: The liquid is orange, and the pellet is a little smaller pellet but visible.
6. Phage w/both Ethidium Bromide and Propidium Iodide: the liquid is bright orange, the pellet is died red and is similar in size to tube 5.
7. Phage w/DRED/Ethidium Bromide/Propidium Iodide: the darkest pellet so far, with a liquid similar to tube 6
8. 50° C. Dyed phage w/DirectRed81: The pellet appears darker than the unheated DRED only tube.
9. 50° C. Phage w/Ethidium Bromide: black ball is what is observed as the pellet the liquid is bright red compared to the room temperature, pellet it is much darker. Not evenly dispersed pellet.
10. 50° C. Phage w/Propidium Iodide: Bright red pellet, much darker than the room temperature.
11. 50° C. Phage w/both Ethidium Bromide and Propidium Iodide: very similar pellet to the unheated tube 6.
12. 50° C. Phage w/DRED/Ethidium Bromide/Propidium Iodide: very dark pellet similar to the pellet in tube 7.

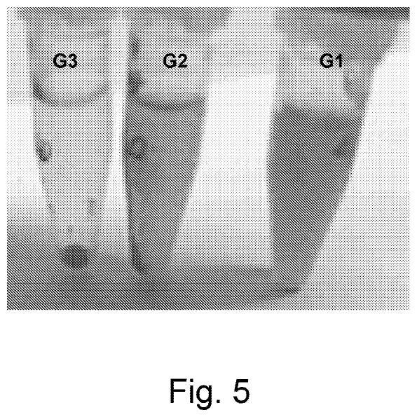

FIG. 5 illustrates tubes containing Ghost particles with DRED and ProID (G3), Ghost particles with DRED and EthBr (G2), and Ghost particles with DRED (G1) which were formed as described below.
1. DRED Ghost particles: nearly no pellet if anything
   a. Ghost particles held at 50° C. for 1 hr in saturated DRED
   b. Phage pelleted and suspended in phage buffer
2. DRED/Ethidium Ghost particles: large pellet going up the side of the tube, very dark
   a. Ghost particles held at 50° C. for 1 hr in saturated DRED/Ethidium
   b. Phage pelleted and suspended in phage buffer
3. DRED/Propidium Iodide Ghost particles: liquid is clear, the pellet is dark and travels up the side of the tube
   a. Ghost particles held at 50° C. for 1 hr in saturated DRED/Propidium Iodide
   b. Phage pelleted and suspended in phage buffer

After all the tubes are made and tested using via NANODROP® microvolume spectrophotometer (available from NanoDrop Technologies LLC of Wilmington, Delaware), a dilution series is made for all of the tubes as follows:
1. Using 250 microliter PCR tubes place 100 microliters of phage buffer in each tube (8 tubes for each original tube and 8 tubes for each supernatant).
2. Then complete a dilution series for each using 100 microliters from each initial tube.
3. Using solubilizing antigen removing buffer SARB-1, make each plate for each dilution series.
   a. Using 5 microliters, do a spot test for each series.
      Table 4 below sets for the results of spot tests for various formulations.

TABLE 4

| | Titer on SARB-1or | Titer of Supernatant |
|---|---|---|
| Tube Number | 2 Spot Test | on SARB-1 Spot Test |
| 1-Undyded Det-7 | −8 Spot (Unreadable) | No Spot |
| 2-Undyded Ghosts | −5 Spot (Unreadable) | No Spot (Bad Lawn) |
| 3-Det-7 and DRED | 1.8 × 10⁹ | 7.4 × 10⁷ |
| 4-Det-7 and EthBr | −8 Spot (Unreadable) | No Spot |
| 5-Det-7 and ProId | 1.2 × 10⁸ | −8 spot (Bad Lawn) |
| 6-Det-7 and EthBr&ProId | −8 Spot (Unreadable) | −8 Spot (Bad Lawn) |
| 7-Det-7 and DRED, EthBr, ProId | 3.2 × 10⁸ | 1.0 × 10⁷ |
| 8-Det-7 @ 50 and DRED | −8 spot (unreadable) | 3.2 × 10⁷ |
| 9-Det-7 @50 and EthBr | No spots | No spots |
| 10-Det-7 @50 and ProId | 9 × 10⁸ (unreadable) | 2.0 × 10⁶ |
| 11-Det-7 @50 and EthBr&ProId | No spots | No spots |
| 12-Det-7 @50 and DRED, EthBr, ProId | 3.14 × 10⁶ | 6.6 × 10⁵ |
| G1-Ghost and DRED | | |
| G2-Ghost and DRED & EthBr | | |
| G3-Ghost and DRED & ProId | | |

In general, any photoacoustic tag or label is suitable for use in the present devices, systems, and methods. Other representative examples include fluorescein isothiocyanate, Evans blue dye, IR775S, Blue and Direct Red 81.

Production of bacteriophage tails for attachment to microspheres. Bacteriophage were purified using cesium chloride (CsCl) gradient purification. Osmotic shock was used to removed phage capsids from tails. Bacteriophage were produced and concentrated to be 1×10¹² pfu/ml and CsCl was added to increase the density of the solution to 1.5 g/ml. Bacteriophage were incubated in CsCl overnight allowing the CsCl to infuse into the bacteriophage DNA that is tightly packaged in the capsid. CsCl infused bacteriophage were then rapidly diluted into phage buffer, causing the rapid diffusion of CsCl out of the bacteriophage. This rapid diffusion resulted in the separation of bacteriophage capsid and tails at the neck connecter. This process of separating intact capsids and tails has been used by phage biologists for many years.

Phage tails were further purified using Bio-Rad HPLC (Hercules, California) and purity was determined by spectrophotometry as well as electron microscopy. Protein purity and concentration was calculated from optical absorbance using a BioTek Synergy H1 (Winooski, Vermont). A 96 well plate was used with the BioTek Synergy H1 to obtain multiple optical absorbance measurements at 280 nm. The Beer-Lambert law was then used to estimate the protein concentration. The absorbance ration between 260 nm and 280 nm was then used to estimate the purity of our protein and any possible DNA contamination.

Electron micrographs were taken on a THermo Fisher/FEI T12 Spirit using a Gatan US 1000 and Orius CCD camera (Hillsboro, Oregon). Micrographs were examined to for the presence of contaminating DNA or groEL, both of which commonly purify with bacteriophage. Tail preparations were found to be of high quality and purity with no observation of contaminating DNA or groEL. Following confirmation of purity, tail preparations were used in later procedures for attachment to microspheres.

Attachment of Phage Tails to Streptavidin Coated Microspheres. Streptavidin coated dyed polystyrene microspheres with nominal diameter of 0.19 μm were obtained from Bangs Laboratories (Fishers, Indiana). Streptavidin coated microspheres were washed four times in PBS to remove stabilizer and antimicrobial agents used by the manufacturer. Microspheres were washed using PBS and using Spin-X concentrator columns three times. Biotin (Thermo Scientific EZ-Link Sulfo-NHS-Biotin) was prepared separately and resuspended in PBS at a concentration of at least 20-fold excess to the phage tail protein binding. Biotin and purified tails were combined and incubated on ice for two hours. After incubation, excess biotin was removed by dialysis using 2 kD molecular weight cut off dialysis cassettes (Slide-A-Lyzer, Thermo Scientific). Biotinilated tails were incubated with washed streptavidin coated microspheres at room temperature for 30 minutes with gentle mixing. Microspheres were washed ten times to remove any unbound biotinylated phage tails. Microspheres with bound phage tails were then concentrated using slow speed centrifugation.

Verification of Functionalized Probes. Microspheres with attached tails were examined using electron microscopy. Samples were negatively stained and multiple dilutions of microsphere with bound tails were examined. Uniform attachment of tails was observed. No microspheres were identified that did not have a full complement of unbound phage tails. Very few free-floating unbound phage tails were identified in each preparation suggesting that both the utilized tail binding and washing procedures were effective. Additionally, signs of contamination by DNA or groEL were investigated. No presence of either was observed in any prepared samples suggesting a high level of purity. Examples of functionalized microspheres can be seen in FIGS. 6A and 6B.

Photoacoustic Flow Cytometry using Functionalized Microspheres. Overnight cultures of each Staph aureus strain were prepared in Mannitol Salt Phenol Broth (MSB) media (Millipore Sigma, Burlington, MA). Overnight cultures were diluted 1/20 and regrown in LB media for two hours to ensure synchronous cultures in exponential growth phase. After re-growth cultures were pelleted and diluted to desired concentration for PAFC. Each sample was diluted to contain roughly 100 bacterial cells per test. Functionalized microspheres were added to diluted bacterial cultures and incubated at room temperature for 10 minutes to allow binding to bacterial surfaces. An excess of functionalized microspheres was added to each bacterial culture so that there were approximately 500 functionalized microspheres per bacterial cell. Combined samples were run on PAFC system using two-phase flow at a combined rate of 60 μl/min.

8| To test the functionalized microspheres for binding and signal generation two bacterial strains were used, Salmonella LT2 is the target host for bacteriophage Det7 from which the tails were produced. Specificity of binding and host range of bacteriophage Det7 has previously been established. As a negative binding control, E. coli strain K12 was used to which bacteriophage Det7 does not bind. Black 1 μm polystyrene microspheres were tested in the PAFC system as a positive control and to give high detection signals. As a negative control, it was first demonstrated that zero signals were produced from our resuspension buffer PBS. Subsequently, both bacterial strains were run at concentrations equal to our testing concentrations demonstrating zero signals. For this series of experiments streptavidin coated red and blue dyed 0.2 μm polystyrene microspheres were used. Each color microsphere was tested at 1× and 10× concentrations. Zero detections were registered for either color regardless or concentration.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A method of determining antibiotic resistance of bacteria, comprising:
dividing a sample including the bacteria into a plurality of sub-samples;
mixing labeling agents, wherein each of the labeling agents is detectible via a detection system including a sensor to detect photoacoustic waves, with each of the plurality of sub-samples, the labeling agents being active to selectively bind with the bacteria;
applying an antibiotic to one or more of the plurality of sub-samples and not to one or more others of the plurality of sub-samples;
characterizing at least one of the sub-samples to which the antibiotic has been applied and at least one of the sub-samples to which no antibiotic has been applied after a determined period of time via the detection system; and
determining antibiotic resistance of the bacteria on the basis of the characterization via the detection system.

2. The method of claim 1 wherein characterization via the detection system comprises quantifying the bacteria in the at least one of the sub-samples to which the antibiotic has been applied and the at least one of sub-samples to which no antibiotic has been applied after the determined period of time.

3. The method of claim 2 wherein determining antibiotic resistance of the bacteria comprises characterizing a difference between the quantification of the bacteria in the at least one of the sub-samples to which the antibiotic has been applied and the quantification of the bacteria in the at least one of sub-samples to which no antibiotic has been applied after the determined period of time.

4. The method of claim 3 wherein determining antibiotic resistance of the bacteria comprises making a determination if the bacteria is resistant to the antibiotic or homogeneous susceptible to the antibiotic.

5. The method of claim 3 wherein determining antibiotic resistance of the bacteria comprises making a determination if the bacteria is homogeneous resistant, heterogeneous resistant or homogeneous susceptible to the antibiotic.

6. The method of claim 1 wherein the labeling agents comprise one or more labels that are detectible via the detection system attached to an entity which is active to selectively bind to the bacteria.

7. The method of claim 6 wherein the entity comprises a protein, a peptide, a bacteriophage, or a portion of a bacteriophage.

8. The method of claim 7 wherein the portion of a bacteriophage is a bacteriophage ghost, a bacteriophage from which at least a portion of a capsid thereof has been removed, a section of a bacteriophage including one or more host-recognition proteins, or a bacteriophage host-recognition protein.

9. The method of claim 6 wherein the one or more labels comprise a compound that is detectible via the detection system attached to the entity.

10. The method of claim 6 wherein the one or more labels comprise a solid particle that is detectible via a detection system attached to the entity, wherein the solid particle is between 1 nm and 500 μm in size, 10 nm and 500 μm in size, or 10 nm and 100 μm in size.

11. The method of claim 10 wherein a plurality of the entities are attached to the solid particle.

12. The method claim 6 wherein the antibiotic comprises an aminoglycoside, a carbapenems, acephalosporins, a quinolone, a fluoroquinolone, a glycopeptide, a lipoglycopeptide, a macrolide, an oxazolidinones, a penicillin, a polypeptide, a rifamycin, a sulfonamide, a streptogramins, a tetracyclines, chloramphenicol, clindamycin, daptomycin, fosfomycin, lefamulin, metronidazole, mupirocin, nitrofurantoin, or tigecycline.

13. The method of claim 12 wherein the entity is a bacteriophage ghost, a bacteriophage from which at least a portion of a capsid thereof had been removed, a section of a bacteriophage including a host-recognition protein, or a host-recognition protein of a bacteriophage.

14. The method of claim 7 wherein the portion of a bacteriophage is a bacteriophage from which at least a portion of the capsid thereof has been removed and a section of a bacteriophage including one or more host-recognition proteins.

15. The method of claim 6 wherein a sufficient number of labeling agents is mixed with the bacteria so that at least one labeling agent is bound to each cell of the bacteria.

16. The method of claim 6 wherein a sufficient number of the labeling agents is mixed with the bacteria so that a plurality of labeling agents is bound to each cell of the bacteria.

17. The method of claim 6 wherein the bacteriophage has been modified to delay or eliminate lysing of a bacterial cell and is active to selectively bind to a species of bacteria

18. A labeling agent comprising one or more labels that are detectible via a detection system including a sensor to detect photoacoustic waves attached to at least a portion of a bacteriophage which has been modified to delay or eliminate lysing of a bacterial cell and is active to selectively bind to a species of bacteria.

19. The labeling agent of claim 18 wherein the one or more labels are attached to a portion of a bacteriophage, and wherein at least a portion of bacteriophage nucleic acid is absent from the portion of a bacteriophage.

20. The labeling agent of claim 19 wherein the portion of a bacteriophage comprises a bacteriophage ghost, a bacteriophage from which at least a portion of a capsid thereof has been removed, a section of a bacteriophage including one or more host-recognition proteins, or a host-recognition protein.

21. The labeling agent of claim 18 wherein the one or more labels comprise a compound that is detectible via the detection system.

22. The labeling agent of claim 19 wherein the one or more labels comprise a solid particle that is detectible via the detection system, wherein the solid particle is between 1 nm and 500 μm in size, 10 nm and 500 μm in size, or 10 nm and 100 μm in size.

23. The labeling agent of claim 22 wherein the solid particle is attached to a plurality of the portions of a bacteriophage.

24. The labeling agent of claim 22 wherein the portion of a bacteriophage is selected from the group consisting of a bacteriophage ghost, a bacteriophage from which at least a portion of the capsid thereof had been removed, a section a bacteriophage including one or more host-recognition proteins, and a host-recognition protein of a bacteriophage.

25. The labeling agent of claim 22 wherein the portion of a bacteriophage is selected from the group consisting of a bacteriophage from which at least a portion of a capsid thereof had been removed and a portion of a bacteriophage tail including one or more host-recognition proteins.

26. The labeling agent of claim 22 wherein the solid particle is inherently detectible via the detection system or comprises one or more entities that are detectible via a detection system.

27. A method of labeling bacteria for detection via a photoacoustic detection system including a sensor to detect photoacoustic waves, comprising: attaching to the bacteria a labeling agent comprising one or more labels that are detectible via the detection system, the one or more labels being attached to a bacteriophage which has been modified to delay or eliminate lysing of a bacterial cell and is active to selectively bind to a species of bacteria or to a portion of a bacteriophage which is active to selectively bind to a species of bacteria.

28.-51. (canceled)
